(19) United States
(12) Patent Application Publication
(10) Pub. No.: US 2025/0331789 A1
(43) Pub. Date: Oct. 30, 2025

(54) GANTRY FOR A COMPUTED TOMOGRAPHY DEVICE WITH WARNING LAMP

(71) Applicant: Siemens Healthineers AG, Forchheim (DE)

(72) Inventors: Christoph EICHNER, Erlangen (DE); Patric HEIBEL, Moschheim (DE); Yenifer HOEGEN, Erlangen (DE); Jens FEHRE, Hausen (DE); Martin RAMSAUER, Pyrbaum (DE); Tobias KOTULLA, Stuttgart (DE)

(73) Assignee: Siemens Healthineers AG, Forchheim (DE)

(21) Appl. No.: 19/190,949

(22) Filed: Apr. 28, 2025

(30) Foreign Application Priority Data
Apr. 29, 2024 (DE) 10 2024 203 984.5

(51) Int. Cl. A61B 6/10; A61B 6/00; A61B 6/03

(52) U.S. Cl. CPC A61B 6/10; A61B 6/032; A61B 6/4405

(57) ABSTRACT

An example embodiment relates to a gantry, the gantry comprising a housing section located, in relation to a radial direction perpendicular to a system axis of the gantry, between a lateral region of an area around the gantry and the system axis; an opening extending in a manner along the system axis of the gantry such that an object under examination can be introduced into the opening along the system axis and can be examined in the opening via X-ray radiation; and a warning lamp for warning of the X-ray radiation, the warning lamp extending along a longitudinal axis of the warning lamp, the warning lamp being on the housing section such that the longitudinal axis of the warning lamp is parallel or substantially parallel to the system axis of the gantry and the warning lamp is visible from the lateral region of the area around the gantry.

### CROSS-REFERENCE TO RELATED APPLICATION(S)

The present application claims priority under 35 U.S.C. § 119 to German Patent Application No. 10 2024 203 984.5, filed Apr. 29, 2024, the entire contents of which is incorporated herein by reference.

### RELATED ART

In an imaging examination using a computed tomography device, X-ray radiation is emitted during a scan. A warning lamp is used to warn of the presence of the X-ray radiation. During certain imaging examinations, particularly with stationary computed tomography devices, the medical personnel performing the examination are usually located in a control room outside the examination room and have a clear view, through a window, of the computed tomography device, typically of an examination opening of the computed tomography device. In these cases, it is sufficient for the warning lamps to be installed on the front and/or rear of the computed tomography device and to be visible from the control room. For example, the warning lamps can be incorporated in the front and/or rear paneling in the form of button lights. In these locations, however, the warning lamps are not visible from every position in the examination room.

Particularly in the case of imaging examinations using mobile computed tomography devices, medical personnel may be present inside the examination room, with various areas within the examination room being possible locations for medical personnel during the scan. For this reason, the warning lamp should be visible from as many areas of the examination room as possible.

### SUMMARY

One or more example embodiments provides a gantry for a computed tomography device, said gantry ensuring improved visibility of an x-ray radiation warning lamp. Independent of the grammatical term usage, individuals with male, female or other gender identities are included within the term.

### BRIEF DESCRIPTION OF THE DRAWINGS

Example embodiments will now be explained by way of example with reference to the accompanying figures. The illustrations in the figures are schematic, highly simplified, and not necessarily to scale.

FIG. 1 shows a front view of a gantry of a computed tomography device according to one or more example embodiments.

FIG. 2 shows a first side view of the gantry of the computed tomography device according to one or more example embodiments.

FIG. 3 shows a rear view of the gantry of the computed tomography device according to one or more example embodiments.

FIG. 4 shows a second side view of the gantry of the computed tomography device according to one or more example embodiments.

FIG. 5 shows a computed tomography device according to one or more example embodiments.

FIG. 6 shows a sectional view of a warning lamp according to one or more example embodiments.

### DETAILED DESCRIPTION

One or more example embodiments relates to a gantry for a computed tomography device,
wherein the gantry comprises a housing section, an opening, and a warning lamp for warning of the presence of X-ray radiation,
wherein the opening extends in a tunnel-like manner along a system axis of the gantry, such that an object under examination can be introduced into the opening along the system axis and examined within the opening using X-ray radiation,
wherein the housing section, in relation to a radial direction perpendicular to the system axis of the gantry, is located between a lateral region of an area around the gantry and the system axis,
wherein the warning lamp extends along a longitudinal axis of the warning lamp, the warning lamp being arranged on the housing section such that the longitudinal axis of the warning lamp is essentially parallel to the system axis of the gantry and that the warning lamp is visible from the lateral region of the area around the gantry.

In particular, the warning lamp can be arranged on the housing section such that the warning lamp is visible from the lateral region of the area around the gantry to a person looking at the gantry from the lateral region of the area around the gantry. In particular, the warning lamp can be arranged on the housing section in such a way that the warning lamp is visible from the lateral region of the area around the gantry in a lateral line of sight, wherein the lateral line of sight is perpendicular to the longitudinal axis of the warning lamp. In particular, it can be provided that the lateral line of sight and the radial direction are coplanar and/or lie in a plane perpendicular to the system axis. In particular, it can be provided that the housing section is located, in relation to the radial direction, between the lateral region of the area around the gantry and the opening and/or that the housing section extends in a tower-like manner essentially perpendicular to the radial direction.

The gantry can, for example, comprise an X-ray source for generating X-ray radiation. In particular, it can be provided that the gantry comprises a radiation shield that protects the lateral region of the area around the gantry from scattered X-ray radiation emanating from the opening and directed toward the lateral region of the area around the gantry and/or aligned in the radial direction. For example, the radiation protection shield can be located, in relation to the radial direction, which is perpendicular to the system axis of the gantry, between the housing section and the system axis. This ensures that a person looking at the gantry from the lateral region of the area around the gantry is protected from the scattered X-ray radiation.

The warning lamp can, for example, be a spatially continuous structure or consist of a plurality of separate lamp units that are successively arranged along the longitudinal axis of the warning lamp and/or are spaced apart along the longitudinal axis of the warning lamp. In particular, it can be provided that the warning lamp extends essentially in a rod-shaped manner along the longitudinal axis of the warning lamp and/or that the warning lamp is attached to the housing section. Light produced by the warning lamp to warn of the presence of X-ray radiation can, for example, be orange. For example, the gantry can have a control unit that is designed to control the warning lamp in such a way that the warning lamp generates light to warn of the presence of X-ray radiation precisely when X-ray radiation is being generated within the gantry by an X-ray source, for example. The warning lamp can, for example, also be designed to generate another light that is not used to warn of the presence of X-ray radiation, but instead indicates, for example, an X-ray-radiation-free operating state of the gantry and/or information relating to the object under examination. The control unit can be a computer, for example.

In particular, it can be provided that the system axis is horizontal and/or that the radial direction, which is perpendicular to the system axis of the gantry, is horizontal. In particular, it can be provided that the warning lamp is arranged on the housing section in such a way that the longitudinal axis of the warning lamp is essentially horizontal. In particular, it can be provided that, in at least one operating state of the gantry in which an extension of the gantry along the system axis is minimal, the extension of the warning lamp along the system axis amounts to more than 25%, for example more than 50%, of the extension of the gantry along the system axis.

One embodiment provides that the warning lamp has a first longitudinal light exit region that extends along the longitudinal axis of the warning lamp, wherein the warning lamp is arranged on the housing section in such a way that the first longitudinal light exit region is visible from the lateral region of the area around the gantry.

In particular, the warning lamp can be arranged on the housing section such that the first longitudinal light exit region is visible in a side view of the gantry from the lateral region of the area around the gantry, wherein a projection direction of the side view of the gantry is perpendicular to the longitudinal axis of the warning lamp and/or to the system axis. The first longitudinal light exit region can, for example, be spatially continuous or consist of a plurality of separate sub-regions that are arranged sequentially along the longitudinal axis of the warning lamp and/or are spaced apart along the longitudinal axis of the warning lamp. The first longitudinal light exit region can, for example, be a first longitudinal light exit surface and/or extend in a planar manner along the longitudinal axis of the warning lamp. The first longitudinal light exit surface can, for example, be curved, in particular curved about the longitudinal axis of the warning lamp.

One embodiment provides that the first longitudinal light exit region is flush-mounted to a surface of the housing section, said surface facing the lateral region of the area around the gantry, in particular such that a gap between the first longitudinal light exit region and the surface of the housing section, said surface facing the lateral region of the area around the gantry, extends essentially parallel to the longitudinal axis of the warning lamp.

One embodiment provides that the warning lamp has a first end-face light exit region which intersects the longitudinal axis of the warning lamp, wherein the warning lamp is arranged on the housing section such that the first end-face light exit region is visible from a front region of the area around the gantry in a first axial line of sight, wherein the first axial line of sight is parallel to the longitudinal axis of the warning lamp.

In particular, the warning lamp can be arranged on the housing section such that the first end-face light exit region is visible in a front view of the gantry from the front region of the area around the gantry, wherein a projection direction of the front view of the gantry is parallel to the longitudinal axis of the warning lamp and/or to the system axis. The first end-face light exit region may, for example, be spatially continuous or composed of a plurality of separate sub-regions that are arranged side by side perpendicular to the longitudinal axis of the warning lamp and/or are spaced apart perpendicular to the longitudinal axis of the warning lamp. The first end-face light exit region can, for example, be a first end-face light exit surface. The first end-face light exit surface can, for example, be curved, in particular curved about a curvature axis of the first end-face light exit surface, said axis being perpendicular to the longitudinal axis of the warning lamp. In particular, it can be provided that the first longitudinal light exit region and the first end-face light exit region transition seamlessly and/or continuously into one another and/or are formed integrally with one another.

In particular, it can be provided that the warning lamp has a second end-face light exit region which intersects the longitudinal axis of the warning lamp, wherein the warning lamp is arranged on the housing section in such a way that the second end-face light exit region is visible from a rear region of an area around the gantry in a second axial line of sight, wherein the second axial line of sight is parallel to the longitudinal axis of the warning lamp and opposite the first axial line of sight. In particular, it can be provided that the warning lamp extends along the longitudinal axis of the warning lamp from the first end-face light exit region to the second end-face light exit region.

In particular, the warning lamp can be disposed on the housing section such that the second end-face light exit region is visible in a rear view of the gantry from the rear region of the area around the gantry, wherein a projection direction of the rear view of the gantry is parallel to the longitudinal axis of the warning lamp and/or to the system axis. In particular, the projection direction of the rear view of the gantry can be opposite the projection direction of the front view of the gantry. The second end-face light exit region may, for example, be spatially continuous or composed of a plurality of separate sub-regions that are arranged side by side perpendicular to the longitudinal axis of the warning lamp and/or are spaced apart perpendicular to the longitudinal axis of the warning lamp.

The second end-face light exit region can, for example, be a second end-face light exit surface. The second end-face light exit surface can, for example, be curved, in particular curved about a curvature axis of the second end-face light exit surface, said curvature axis being perpendicular to the longitudinal axis of the warning lamp. In particular, it can be provided that the first longitudinal light exit region and the second end-face light exit region transition seamlessly and/or continuously into one another and/or are formed integrally with one another.

One embodiment provides that the first end-face light exit region is flush-mounted to a surface of the housing section, said surface facing a front region of the area around the gantry, in particular such that a gap between the first end-face light exit region and the housing section surface facing the front region of the area around the gantry extends essentially perpendicular to the longitudinal axis of the warning lamp.

In particular, it can be provided that the second end-face light exit region is flush-mounted to a surface of the housing section, said surface facing a rear region of the area around the gantry, in particular such that a gap between the second end-face light exit region and the housing section surface facing the rear region of the area around the gantry extends essentially perpendicular to the longitudinal axis of the warning lamp.

One embodiment provides that the warning lamp has an upper light exit region that extends along the longitudinal axis of the warning lamp, wherein the warning lamp is arranged on the housing section such that the upper light exit region is visible in a top view of the gantry. In particular, the warning lamp can be arranged on the housing section in such a way that the upper light exit region is visible from an upper region of the area around the gantry and/or that the upper light exit region lies within a field of view of a camera. The camera can, for example, be arranged on a ceiling and/or above the gantry.

A projection direction of the top view of the gantry can, for example, be vertical and/or perpendicular to the longitudinal axis of the warning lamp and/or to the system axis. The upper light exit region can, for example, be an upper light exit surface. The upper light exit surface can, for example, be curved, in particular curved about a curvature axis of the upper light exit region, said curvature axis being perpendicular to the longitudinal axis of the warning lamp. In particular, it can be provided that the first longitudinal light exit region and the upper light exit region transition seamlessly and/or continuously into one another and/or are formed integrally with one another, and/or that the first end-face light exit region and the upper light exit region transition seamlessly and/or continuously into one another and/or are formed integrally with one another, and/or that the second end-face light exit region and the upper light exit region transition seamlessly and/or continuously and/or are formed integrally with one another.

One embodiment provides that a contact surface of the warning lamp extends in a planar manner along the longitudinal axis of the warning lamp, wherein the warning lamp is arranged on the housing section such that the contact surface of the warning lamp lies flat against a contact surface of the housing section. The contact surface of the housing section can, for example, be horizontal and/or parallel to the system axis. In particular, the warning lamp can be arranged on the housing section in such a way that the contact surface of the warning lamp is parallel to the contact surface of the housing section and/or that the contact surface of the warning lamp is horizontal and/or parallel to the system axis.

One embodiment provides that the warning lamp has a protrusion relative to the contact surface of the warning lamp, said protrusion extending along the longitudinal axis of the warning lamp, wherein the housing section has a recess relative to the contact surface of the housing section, said recess extending essentially parallel to the system axis, wherein the warning lamp is arranged on the housing section such that the protrusion is received in the recess in a form-fitting manner.

In particular, it can be provided that the warning lamp has a lamp-side connecting element, that the housing section has a housing-side connecting element, and that a connection is established via the lamp-side connecting element and the housing-side connecting element, which connection counteracts removal of the protrusion from the recess.

The housing-side connecting element can, for example, be a screw. The lamp-side connecting element can, for example, be a screw nut and/or have a thread for the screw. There can, for example, be a cavity within the protrusion. A cable of the warning lamp and/or an electronic component of the warning lamp can, for example, be located within said cavity.

One embodiment provides that the housing section comprises paneling for separating the housing section interior from the area around the gantry, wherein the paneling is opaque to light generated by the warning lamp.

In particular, it can be provided that the paneling comprises the housing section surface facing the lateral region of the area around the gantry and/or the housing section surface facing the front region of the area around the gantry. The paneling can comprise, for example, the contact surface of the housing section and/or the recess.

One embodiment provides that that the warning lamp comprises a cover for separating the interior of the warning lamp from the area around the gantry, wherein the cover is translucent to light generated by the warning lamp. In particular, for light generated by the warning lamp, the cover of the warning lamp can be translucent and the paneling of the housing section can be opaque. In particular, it can be provided that the cover comprises the first longitudinal light exit region and/or the second longitudinal light exit region. In particular, it can be provided that the cover comprises the first end-face light exit region and/or the second end-face light exit region. In particular, it can be provided that the cover comprises the upper light exit region.

In particular, the cover can be designed as a diffuser for the light generated by the warning lamp. The cover can, for example, be made of polycarbonate and/or polymethylmethacrylate and/or produced by injection molding. In particular, the cover of the warning lamp can be painted and/or coated and/or have surface patterning such that the cover of the warning lamp essentially matches the paneling of the housing section in terms of color impression and/or gloss effect, in particular, matches it in such a way that, when the warning lamp is switched off, the cover of the warning lamp and the paneling of the housing section are visually almost indistinguishable. In particular, it can be provided that the cover of the warning lamp and the paneling of the housing section appear in the same color tone, for example in the same shade of white, when the warning lamp is switched off. The patterned surface of the cover can be achieved, for example, via a correspondingly patterned surface of an injection mold designed to produce the cover.

One embodiment provides that the gantry comprises a first gantry part and a second gantry part, wherein the first gantry part comprises an X-ray source for generating X-ray radiation and is mounted such that it can move relative to the second gantry part via a linear guide in such a way that a translational movement of the first gantry part relative to the second gantry part can be performed along the system axis, wherein the first gantry part annularly encloses the opening, wherein the second gantry part comprises the housing section and the warning lamp.

In particular, it can be provided that, during the translational movement of the first gantry part relative to the second gantry part along the system axis, the second gantry part remains stationary relative to the lateral region of the area around the gantry. In particular, it can be provided that the linear guide comprises a rail system and a carriage system operating in conjunction with the rail system, wherein the first gantry part comprises the carriage system and the second gantry part comprises the rail system.

In particular, it can be provided that the rotor comprises an X-ray detector for detecting the X-ray radiation. The first gantry part may, for example, comprise rear paneling of the gantry, said rear paneling annularly enclosing a rear side of the opening. The gantry may, for example, have a third gantry part, wherein the third gantry part comprises front paneling of the gantry, wherein said front paneling annularly encloses a front side of the opening.

One embodiment provides that the warning lamp is arranged on the housing section in such a way that the warning lamp does not extend beyond the highest point of the first gantry part. This prevents the height of the gantry—which is important, for example, when passing through doorways—from being increased due to the arrangement of the warning lamp.

One embodiment provides that the warning lamp comprises a second longitudinal light exit region extending along the longitudinal axis of the warning lamp, wherein the warning lamp is arranged on the housing section in such a way that the first gantry part blocks a view of the second longitudinal light exit region in a line of sight passing through a room region when the first gantry part projects into said room region, and that the first gantry part clears the view of the second longitudinal light exit region in the line of sight passing through the room region when the first gantry part exits the room region due to a first translational movement of the first gantry part relative to the second gantry part along the system axis.

One or more example embodiments further relates to a method for operating such a gantry, wherein the first gantry part blocks the view of the second longitudinal light exit region in a line of sight passing through the room region when the first gantry part protrudes into the room region, wherein the view of the second longitudinal light exit region in said line of sight is cleared by the first gantry part by performing the first translational movement of the first gantry part relative to the second gantry part along the system axis in such a way that the first gantry part exits the room region.

The second longitudinal light exit region can, for example, be spatially continuous or be composed of a plurality of separate sub-regions that are arranged sequentially along the longitudinal axis of the warning lamp and/or are spaced apart along the longitudinal axis of the warning lamp.

The second longitudinal light exit region can, for example, be a second longitudinal light exit surface and/or extend in a planar manner along the longitudinal axis of the warning lamp. The second longitudinal light exit surface can, for example, be curved, in particular curved about the longitudinal axis of the warning lamp. In particular, it can be provided that, during the first translational movement of the first gantry part relative to the second gantry part along the system axis, the second gantry part remains stationary relative to the room region and/or relative to the lateral region of the area around the gantry. In particular, it can be provided that, during the first translational movement of the first gantry part relative to the second gantry part along the system axis, the third gantry part remains stationary relative to the first gantry part.

In particular, it can be provided that the lateral region of the area around the gantry is a first lateral region of the area around the gantry, that the warning lamp is arranged on the housing section such that the first longitudinal light exit region is visible from the first lateral region of the area around the gantry in a first side view of the gantry, wherein a projection direction of the first side view of the gantry is perpendicular to the longitudinal axis of the warning lamp and/or to the system axis. In particular, it can be provided that the first gantry part clears the view of the second longitudinal light exit region in the line of sight passing through the room region in such a way that, when the first gantry part exits the room region due to the first translational movement of the first gantry part relative to the second gantry part along the system axis, the part of the second longitudinal light exit region which is visible from a second lateral region of the area around the gantry in a second side view of the gantry increases, wherein a direction of projection of the second side view of the gantry is perpendicular to the longitudinal axis of the warning lamp and/or to the system axis and is opposite to the direction of projection of the first side view of the gantry.

In preparation for an imaging examination, the first translational movement can be performed, for example, prior to the X-ray radiation being triggered. Thus, the portion of the second longitudinal light exit region that is visible from the second lateral region of the area around the gantry in the second side view of the gantry is greatest immediately before the X-ray radiation is triggered.

In particular, it can be provided that the warning lamp is arranged on the housing section in such a way that the first gantry part does not block the view of the second longitudinal light exit region in the line of sight passing through the room region when the first gantry part is located outside the room region, and that the first gantry part blocks the view of the second longitudinal light exit region in the line of sight passing through the room region to the extent to which the first gantry part enters the room region as a result of a second translational movement of the first gantry part relative to the second gantry part along the system axis.

In particular, it can be provided that, during the second translational movement of the first gantry part relative to the second gantry part along the system axis, the third gantry part remains stationary relative to the second gantry part. Particularly when the warning lamp generates light, a position of the first gantry part relative to the second gantry part and/or progression of the imaging examination can thus be displayed, particularly in the form of an illuminated progress bar. The line of sight passing through the room region can be horizontal and/or perpendicular to the system axis.

One or more example embodiments also relates to a computed tomography device comprising a gantry according to one or more example embodiments. The computed tomography device may, for example, be mobile and/or be a head-scan computed tomography device.

In the context of the invention, features described in connection with different embodiments of the invention and/or different claim categories (process, use, apparatus, system, arrangement, etc.) can be combined to create further embodiments of the invention.

For example, a claim relating to an apparatus can also be further developed with features described or claimed in connection with a method and vice versa. Functional features of a method can be implemented by correspondingly designed physical components. The use of the indefinite article “a” or “an” does not exclude the possibility that the feature concerned may also be present more than once. The term “based on” can be understood in the context of the present application in particular as meaning “using”.

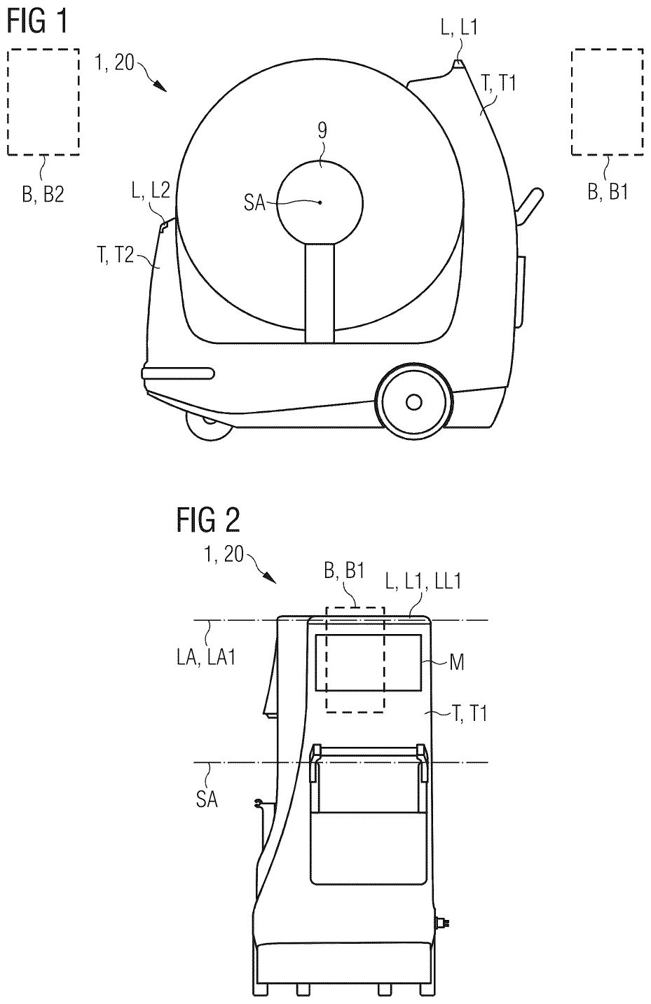

FIG. 1 shows the gantry 20 of the computed tomography device 1 in a front view. The gantry 20 comprises a housing section T, an opening 9, and a warning lamp L, in particular the warning lamp L1 and/or the warning lamp L2, for warning of X-ray radiation 7, wherein the opening 9 extends in a tunnel-like manner along a system axis SA of the gantry 20 such that an object under examination 14 can be introduced into the opening 9 along the system axis SA and examined in the opening 9 using the X-ray radiation 7, wherein the housing section T is located, with respect to a radial direction perpendicular to the system axis SA of the gantry 20, between a lateral region B of an area around the gantry 20 and the system axis SA.

The warning lamp L extends along a longitudinal axis LA of the warning lamp L, wherein the warning lamp L is arranged on the housing section T such that the longitudinal axis LA of the warning lamp L is essentially parallel to the system axis SA of the gantry 20 and such that the warning lamp L is visible from the lateral region B of the area around the gantry 20. The warning lamp L1 and/or the warning lamp L2 are visible from practically any position in the examination room. The two warning lamps are each seamlessly attached to the paneling V.

The warning lamp L1 extends along a longitudinal axis LA1 of the warning lamp L1, wherein the warning lamp L1 is arranged on the housing section T1 such that the longitudinal axis LA1 of the warning lamp L1 is essentially parallel to the system axis SA of the gantry 20 and the warning lamp L1 is visible from the lateral region B1 of the area around the gantry 20. The warning lamp L2 extends along a longitudinal axis LA2 of the warning lamp L2, wherein the warning lamp L2 is arranged on the housing section T2 such that the longitudinal axis LA2 of the warning lamp L2 is essentially parallel to the system axis SA of the gantry 20 and the warning lamp L2 is visible from the lateral region B2 of the area around the gantry 20.

The warning lamp L comprises a first end-face light exit region which intersects the longitudinal axis LA of the warning lamp L, wherein the warning lamp L is arranged on the housing section T in such a way that the first end-face light exit region is visible from a front region of the area around the gantry 20 in a first axial line of sight, wherein the first axial line of sight is parallel to the longitudinal axis LA of the warning lamp L. The first end-face light exit region is flush-mounted to a surface of the housing section T, said surface facing the front region of the area around the gantry 20, in particular such that a gap between the first light exit region on the front side and the surface of the housing section T, said surface facing the front region of the area around the gantry 20, extends essentially perpendicular to the longitudinal axis LA of the warning lamp L.

FIG. 2 shows the gantry 20 of the computed tomography device 1 in a first side view. The warning lamp L has a first longitudinal light exit region LL1 which extends along the longitudinal axis LA of the warning lamp L, wherein the warning lamp L is arranged on the housing section T in such a way that the first longitudinal light exit region LL1 is visible from the lateral region B of the area around the gantry 20.

The first longitudinal light exit region LL1 is flush-mounted to a surface of the housing section T, said surface facing the lateral region of the area around the gantry 20, in particular is attached in such a way that a gap between the first longitudinal light exit region LL1 and the surface of the housing section T, said surface facing the lateral region B of the area around the gantry 20, extends essentially parallel to the longitudinal axis LA of the warning lamp L. The housing section T comprises a touch-sensitive display M for operating the computed tomography device 1. The touch-sensitive display M faces the lateral region B of the area around the gantry 20.

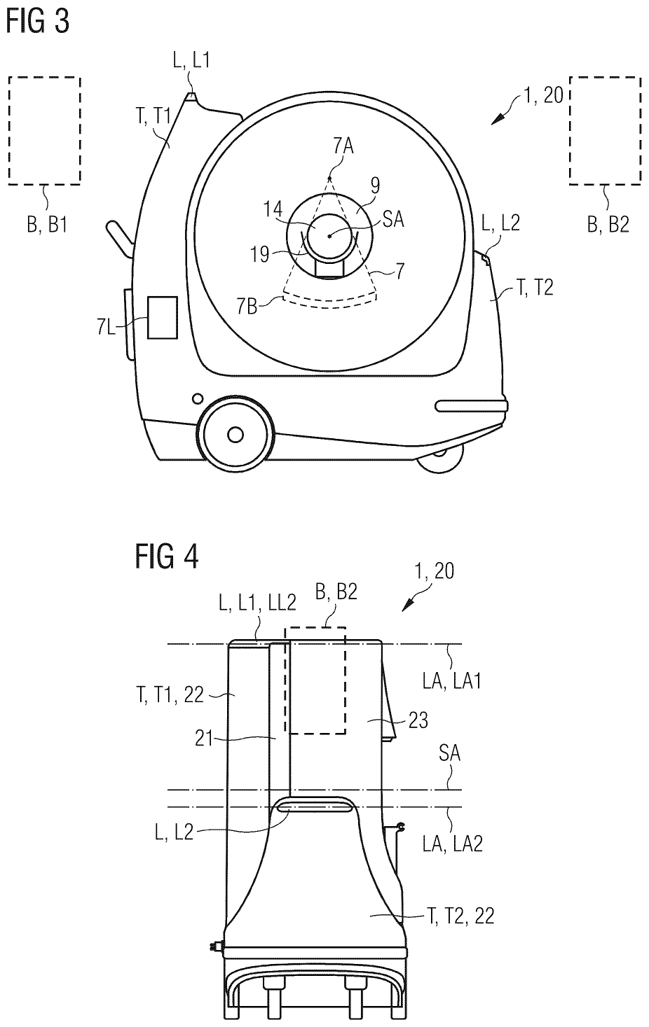

FIG. 3 shows a rear view of the gantry of the computed tomography device.

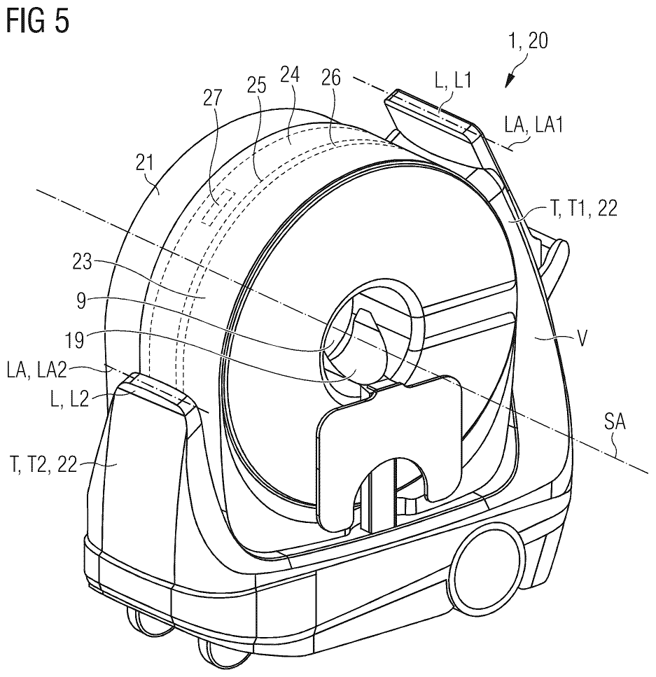

FIG. 4 shows a second side view of the gantry 20 of the computed tomography device 1. The gantry 20 comprises a first gantry part 21 and a second gantry part 22, wherein the first gantry part 21 comprises a rotor 24 with an X-ray source 7A for generating the X-ray radiation 7 and is mounted so as to be movable relative to the second gantry part 22 via a linear guide in such a way that a translational movement of the first gantry part 21 relative to the second gantry part 22 along the system axis SA can be performed, wherein the first gantry part 21 annularly encloses the opening 9, wherein the second gantry part 22 comprises the housing section T and the warning lamp L.

The example shown provides that the rotor 24 comprises an X-ray detector 7B for detecting the X-ray radiation 7. The first gantry part 21 can, for example, comprise rear paneling of the gantry 20, wherein said rear paneling of the gantry 20 annularly encloses a rear side of the opening 9. The gantry 20 can, for example, comprise a third gantry part 23, wherein the third gantry part 23 comprises front paneling of the gantry 20, wherein the front paneling of the gantry 20 annularly encloses a front side of the opening 9. The warning lamp L is arranged on the housing section T in such a way that the warning lamp L does not project beyond the highest point of the first gantry part 21.

The warning lamp L, in particular the warning lamp L1, has a second longitudinal light exit region LL2 which extends along the longitudinal axis LA of the warning lamp L, wherein the warning lamp L is arranged on the housing section T in such a way that the first gantry part 21 blocks a view to the second longitudinal light exit region LL2 in a line of sight passing through a room region, said line of sight originating from the lateral region B2, for example, when the first gantry part 21 projects into the room region, and that the first gantry part 21 clears the view of the second longitudinal light exit region LL2 in the line of sight passing through the room region when the first gantry part 21 exits the room region as a result of a first translational movement of the first gantry part 21 relative to the second gantry part 22 along the system axis SA.

In the example shown, the warning lamp L is arranged on the housing section T such that the first gantry part 21 does not block the view to the second longitudinal light exit region LL2 in the line of sight passing through the room region when the first gantry part 21 is located outside the room region, and that the first gantry part 21 blocks the view to the second longitudinal light exit region LL2 in the line of sight passing through the room region to the extent to which the first gantry part 21 enters the room region as a result of a second translational movement of the first gantry part 21 relative to the second gantry part 22 along the system axis SA.

FIG. 5 shows the computed tomography device 1, comprising the gantry 20 with the opening 9. The computed tomography device 1 is a mobile head-scan computed tomography device. The computed tomography device 1 has a headrest 19 for the object under examination 14, which may, for example, be a human head. The gantry 20 comprises a travel mechanism, wherein the gantry 20 is mounted such that it can move relative to the base, in particular in a horizontally movable manner, via the travel mechanism. The gantry 20 is therefore mobile.

The gantry 20 can, for example, have a direction-of-travel camera, wherein the direction-of-travel camera is arranged on the housing section T2 in such a way that an optical axis of the direction-of-travel camera is essentially perpendicular to the system axis SA. The direction-of-travel camera can, for example, be designed to capture a region of the area around the gantry 20 in front of the housing section T2 in the direction of travel of the travel mechanism, in particular to capture it in such a way that a camera image of this region of the area around the gantry can be shown on the display M. In particular, a person pushing the gantry 20 in the direction of travel of the travel mechanism toward this region of the area around the gantry, wherein the gantry 20 blocks their direct view of this region of the area around the gantry, can observe this region of the area around the gantry using the direction-of-travel camera and the display M. The direction of travel of the travel mechanism can in particular be horizontal and/or perpendicular to the system axis SA. The display M can, for example, be arranged so as to be pivotable relative to the housing section T1 about a pivot axis parallel to the system axis SA.

The direction-of-travel camera can be arranged, for example, in relation to a vertical direction on the housing section T2, between the travel mechanism and the warning lamp L2. The direction-of-travel camera can, for example, be arranged in a cutout in the paneling V, in particular in a recessed manner, particularly in order to avoid parts protruding beyond the paneling V and/or to protect the direction-of-travel camera from collisions.

The first gantry part 21 comprises the rotatably mounted rotor 24 with the projection data acquisition system 27. The projection data acquisition system 27 comprises the X-ray source 7A for generating the X-ray radiation 7 and the X-ray detector 7B for detecting the X-ray radiation 7. The first gantry part 21 comprises the rotary bearing 25 and the support frame 26, wherein the rotor 24 is connected to the support frame 26 via the rotary bearing 25 and is mounted so as to be rotatable relative to the support frame 26 about the system axis SA of the gantry 20. The system axis SA is horizontal.

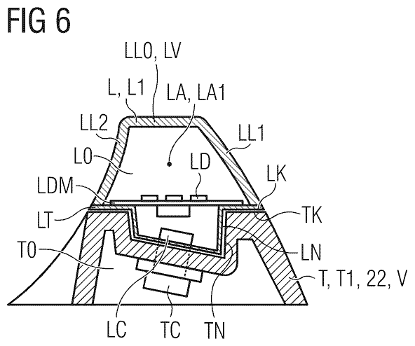

FIG. 6 shows a sectional view of a warning lamp L, in particular of the warning lamp L1, wherein the warning lamp L has an upper light exit region LL0 which extends along the longitudinal axis LA of the warning lamp L, wherein the warning lamp L is arranged on the housing section T in such a way that the upper light exit region LL0 is visible in a top view of the gantry 20. A contact surface LK of the warning lamp L extends in a planar manner along the longitudinal axis LA of the warning lamp L, wherein the warning lamp L is arranged on the housing section T in such a way that the contact surface LK of the warning lamp L lies flat against a contact surface TK of the housing section T.

The warning lamp L has a protrusion LN relative to the contact surface LK of the warning lamp L, said protrusion extending along the longitudinal axis LA of the warning lamp L, wherein the housing section T has a recess TN relative to the contact surface TK of the housing section T, said recess extending essentially parallel to the system axis SA, wherein the warning lamp L is arranged on the housing section T such that the protrusion LN is received in the recess TN in a form-fitting manner.

The example shown provides that the warning lamp L has a lamp-side connecting element LC, that the housing section T has a housing-side connecting element TC, and that the lamp-side connecting element LC and the housing-side connecting element TC establish a connection which counteracts removal of the protrusion LN from the recess TN.

The housing section T comprises paneling V for separating an interior region TO of the housing section T from the area around the gantry 20, wherein the paneling V is opaque to light generated by the warning lamp L. The warning lamp L comprises a cover LV for separating an interior region L0 of the warning lamp L from the area around the gantry 20, wherein the cover LV is translucent to light generated by the warning lamp L.

The warning lamp L has a supporting structure LT, wherein the supporting structure LT comprises the contact surface LK of the warning lamp L and the protrusion LN and is connected to the cover LV of the warning lamp L. For example, pins of the cover LV of the warning lamp L can be inserted into corresponding holes in the supporting structure LT and/or can be welded and/or glued to the supporting structure LT. The supporting structure LT is screwed to the housing section T using a plurality of screw connections arranged sequentially and spaced apart along the longitudinal axis LA of the warning lamp L. The warning lamp L has a light-emitting diode module LDM with a plurality of light-emitting diodes LD. The light-emitting diode module LDM is located in the interior region L0 of the warning lamp L and is connected, for example screwed and/or glued, to the supporting structure LT. The plurality of light-emitting diodes LD can be arranged, for example, in a plurality of rows arranged side by side, each extending along the longitudinal axis LA of the warning lamp L, in particular surface-mounted on a circuit board.

The warning lamp L has a lamp-side interface for power supply and/or data transmission, for example in the form of a socket. The socket can, for example, be arranged on the supporting structure LT and/or formed on the protrusion LN. The housing section T has a housing-side interface for power supply and/or data transmission. The housing-side interface can, for example, be in the form of a plug corresponding to the socket. The plug can, for example, be introduced through a hole in the paneling V of the housing section T, which hole can, in particular, be formed in the recess TN, and/or can be connected to the socket. The plug can be connected to the control unit 7L of the gantry 20 via a cable, for example. The gantry 20 comprises the control unit 7L, which is located in the interior area T0 of the housing section T, for example, and/or which is designed to control the warning lamps L1 and L2, in particular in synchronism with one another, such that the warning lamps L1 and L2 each generate a light to warn of the presence of X-ray radiation 7 precisely when the X-ray radiation 7 is being generated in the gantry 20 via the X-ray source 7A.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems implementing and/or for the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A gantry for a computed tomography device, the gantry comprising:
a housing section located, in relation to a radial direction perpendicular to a system axis of the gantry, between a lateral region of an area around the gantry and the system axis;
an opening extending in a manner along the system axis of the gantry such that an object under examination can be introduced into the opening along the system axis and can be examined in the opening via X-ray radiation; and
a warning lamp for warning of the X-ray radiation, the warning lamp extending along a longitudinal axis of the warning lamp, the warning lamp being on the housing section such that the longitudinal axis of the warning lamp is parallel or substantially parallel to the system axis of the gantry and the warning lamp is visible from the lateral region of the area around the gantry.

2. The gantry of claim 1, wherein
the warning lamp has a first longitudinal light exit region which extends along the longitudinal axis of the warning lamp, and
the warning lamp is on the housing section such that the first longitudinal light exit region is visible from the lateral region of the area around the gantry.

3. The gantry of claim 2, wherein the first longitudinal light exit region is flush-mounted to a surface of the housing section, the surface facing the lateral region of the area around the gantry.

4. The gantry of claim 1, wherein
the warning lamp has a first end-face light exit region which intersects the longitudinal axis of the warning lamp, and
the warning lamp is on the housing section such that the first end-face light exit region is visible from a front region of the area around the gantry in a first axial line of sight, the first axial line of sight being parallel to the longitudinal axis of the warning lamp.

5. The gantry of claim 4, wherein the first end-face light exit region is flush-mounted to a surface of the housing section, the surface facing the front region of the area around the gantry.

6. The gantry of claim 1, wherein
the warning lamp has an upper light exit region which extends along the longitudinal axis of the warning lamp, and
the warning lamp is arranged on the housing section such that the upper light exit region is visible in a top view of the gantry.

7. The gantry of claim 1, wherein
a contact surface of the warning lamp extends in planarly along the longitudinal axis of the warning lamp, and
the warning lamp is on the housing section such that the contact surface of the warning lamp lies flat against a contact surface of the housing section.

8. The gantry of claim 7, wherein
the warning lamp has a protrusion relative to the contact surface of the warning lamp, the protrusion extending along the longitudinal axis of the warning lamp,
the housing section has a recess relative to the contact surface of the housing section, the recess extending parallel or substantially parallel to the system axis, and
the warning lamp is on the housing section such that the protrusion is received in the recess in a form-fitting manner.

9. The gantry of claim 1, wherein
the housing section comprises paneling to separate an interior region of the housing section from the area around the gantry, and
the paneling is opaque to light generated by the warning lamp.

10. The gantry of claim 1, wherein
the warning lamp has a cover to separate an interior region of the warning lamp from the area around the gantry, and
the cover is translucent to light generated by the warning lamp.

11. The gantry of claim 1, wherein
the gantry comprises a first gantry part and a second gantry part,
the first gantry part comprises an X-ray source for generating the X-ray radiation and is mounted using a linear guide so as to be movable relative to the second gantry part such that the first gantry part is translationally moveable relative to the second gantry part along the system axis,
the first gantry part annularly encloses the opening, and
the second gantry part comprises the housing section and the warning lamp.

12. The gantry of claim 11, wherein the warning lamp is on the housing section such that the warning lamp does not project beyond a highest point of the first gantry part.

13. The gantry of claim 11, wherein
the warning lamp has a second longitudinal light exit region extending along the longitudinal axis of the warning lamp, and
the warning lamp is on the housing section such that the first gantry part blocks a view of the second longitudinal light exit region along a line of sight passing through a room region when the first gantry part projects into the room region, and such that the first gantry part clears the view of the second longitudinal light exit region in the line of sight passing through the room region when the first gantry part exits the room region due to a first translational movement of the first gantry part relative to the second gantry part along the system axis.

14. A computed tomography device comprising:
the gantry of claim 1.

15. A method for operating the gantry of claim 13, the method comprising:
using the first gantry part to block the view of the second longitudinal light exit region along the line of sight passing through the room region when the first gantry part projects into the room region; and
using the first gantry part to clear the view of the second longitudinal light exit region along the line of sight passing through the room region when the first translational movement of the first gantry part relative to the second gantry part along the system axis is performed such that the first gantry part exits the room region.

16. The gantry of claim 2, wherein
the warning lamp has a first end-face light exit region which intersects the longitudinal axis of the warning lamp, and
the warning lamp is on the housing section such that the first end-face light exit region is visible from a front region of the area around the gantry in a first axial line of sight, the first axial line of sight being parallel to the longitudinal axis of the warning lamp.

17. The gantry of claim 16, wherein
the warning lamp has an upper light exit region which extends along the longitudinal axis of the warning lamp, and
the warning lamp is arranged on the housing section such that the upper light exit region is visible in a top view of the gantry.

18. The gantry of claim 17, wherein
a contact surface of the warning lamp extends in planarly along the longitudinal axis of the warning lamp, and
the warning lamp is on the housing section such that the contact surface of the warning lamp lies flat against a contact surface of the housing section.

19. The gantry of claim 2, wherein
the housing section comprises paneling to separate an interior region of the housing section from the area around the gantry, and
the paneling is opaque to light generated by the warning lamp.

20. The gantry of claim 19, wherein
the warning lamp has a cover to separate an interior region of the warning lamp from the area around the gantry, and
the cover is translucent to light generated by the warning lamp.
